(19) United States
(12) Patent Application Publication
(10) Pub. No.: US 2022/0170879 A1
(43) Pub. Date: Jun. 2, 2022

(54) TUNABLE NANOPILLAR AND NANOGAP ELECTRODE STRUCTURES AND METHODS THEREOF

(71) Applicant: Roswell Biotechnologies, Inc., San Diego, CA (US)

(72) Inventors: Chulmin Choi, San Diego, CA (US); Sungho Jin, San Diego, CA (US); Paul Mola, San Diego, CA (US)

(73) Assignee: Roswell Biotechnologies, Inc., San Diego, CA (US)

(21) Appl. No.: 17/598,854

(22) PCT Filed: Mar. 26, 2020
(86) PCT No.: PCT/US20/25068

§ 371 Date: Sep. 27, 2021

Related U.S. Application Data
(60) Provisional application No. 62/824,230, filed on Mar. 26, 2019.

(51) Int. Cl. G01N 27/327; C12Q 1/6874

(52) U.S. Cl. CPC G01N 27/3276; G01N 27/3278; C12Q 1/6874

(57) ABSTRACT

New methods in nanolithography provide nanoscale structures usable in molecular electronic sensors, such as for nucleotide sequencing. In various embodiments, tunable nanopillars are grown in holes nanopatterned in a resist layer over pairs of electrodes, with the resulting nanopillars acting as vertical extensions of the electrodes buried underneath the resist layer. Exposed top surfaces of the nanopillars are limited in size, thus providing controlled binding of a single or at most just a few bridge molecules between nanopillars in a pair of nanopillars.

### CROSS-REFERENCE TO RELATED APPLICATIONS

This application claims priority to and the benefit of U.S. Provisional Patent Application Ser. No. 62/824,230 filed Mar. 26, 2019, entitled “TUNABLE NANOPILLAR AND NANO-GAP ELECTRODE STRUCTURES FOR GENOME SEQUENCING,” the disclosure of which is incorporated herein by reference in its entirety for all purposes.

### FIELD

The present disclosure relates to label-free biomolecular sensing devices, and more specifically relates to the formation of dimension-tunable molecular electrodes.

### BACKGROUND

In various fields of precision medicine or nanotechnology, analysis of biomolecules including DNAs and genomes has received an increasing attention in recent years. The seminal work of Maclyn McCarty and Oswald T. Avery in 1946, (see, “Studies On The Chemical Nature Of The Substance Inducing Transformation Of Pneumococcal Types II. Effect Of Deoxyribonuclease On The Biological Activity Of The Transforming Substance,” The Journal of Experimental Medicine 83(2), 89-96 (1946)), demonstrated that DNA was the material that determined traits of an organism. The molecular structure of DNA was then first described by James D. Watson and Francis HC Crick in 1953, (see a published article, “Molecular structure of nucleic acids.”, Nature 171, 737-738 (1953)), for which they received the 1962 Nobel Prize in Medicine. This work made it clear that the sequence of chemical letters (bases) of the DNA molecules encode the fundamental biological information. Since this discovery, there has been a concerted effort to develop means to actually experimentally measure this sequence. The first method for systematically sequencing DNA was introduced by Sanger, et al in 1978, for which he received the 1980 Nobel Prize in Chemistry. See an article, Sanger, Frederick, et al., “The nucleotide sequence of bacteriophage φX174.” Journal of molecular biology 125, 225-246 (1978).

Sequencing techniques for genome analysis evolved into utilizing automated commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Jonathan M. Rothberg's group in 2005 as the 454 platform, which achieved thousand fold reductions in cost and instrument time. See, an article by Marcel Margulies, et al., “Genome Sequencing in Open Microfabricated High Density Picoliter Reactors,” Nature 437, 376-380 (2005). However, the 454 platform still required approximately a million dollars and took over a month to sequence a genome.

The 454 platform was followed by a variety of other related techniques and commercial platforms. See, articles by M. L. Metzker, “Sequencing Technologies—the Next Generation,” Nature reviews genetics 11(1), 31-46 (2010), and by C. W. Fuller et. al, “The Challenges of Sequencing by Synthesis,” Nature biotechnology 27(11), 1013-1023 (2009). This progress lead to the realization of the long-sought “$1,000 genome” in 2014, in which the cost of sequencing a human genome at a service lab was reduced to approximately $1,000, and could be performed in several days. However, the highly sophisticated instrument for this sequencing cost nearly one million dollars, and the data was in the form of billions of short reads of approximately 100 bases in length. The billions of short reads often further contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine (see the aforementioned article by Fuller et al), where it is desirable to sequence the genomes of millions of individuals with a clinical grade of quality.

### SUMMARY

While many DNA sequencing techniques utilize optical means with fluorescence reporters, such methods can be cumbersome, slow in detection speed, and difficult to mass produce to further reduce costs. Label-free DNA or genome sequencing approaches provide advantages of not having to use fluorescent type labeling processes and associated optical systems, especially when combined with electronic signal detection that can be achieved rapidly and in an inexpensive way.

In this regard, certain types of molecular electronic devices can detect single molecule, biomolecular analytes such as DNAs, RNAs, proteins, and nucleotides by measuring electronic signal changes when the analyte molecule is attached to a circuit. Such methods are label-free and thus avoid using complicated, bulky and expensive fluorescent type labeling apparatus. These methods can be useful for lower cost sequencing analysis of DNA, RNA and genome.

Certain types of molecular electronic devices can detect the biomolecular analytes such as DNAs, RNAs, proteins, and nucleotides by measuring electronic signal changes when the analyte molecule is attached to the circuit comprising a pair of conductive electrodes. Such methods are label-free and thus avoids using complicated, bulky and expensive fluorescent type labeling apparatus.

While current molecular electronic devices can electronically measure molecules for various applications, they lack the reproducibility as well as scalability and manufacturability needed for rapidly sensing many analytes at a scale of up to millions in a practical manner. Such highly scalable methods are particularly important for DNA sequencing applications, which often need to analyze millions to billions of independent DNA molecules. In addition, the manufacture of current molecular electronic devices is generally costly due to the high level of precision needed.

Disclosed herein are new and improved sequencing apparatuses, structures and methods using dimension-tunable nanoelectrodes comprising vertical nanopillars or horizontal nanoelectrodes to enable DNA or related elongated bridge structures, which provide reliable DNA genome analysis performance and are amenable to scalable manufacturing.

In various embodiments, a structure for use in a molecular electronics sensor comprises: a pair of nanoelectrodes disposed on a substrate and comprising a first metal, each pair of nanoelectrodes comprising a first nanoelectrode and a second nanoelectrode spaced-apart from the first nanoelectrode by a nanogap; a resist or dielectric layer covering the pair of nanoelectrodes and the nanogap; and a pair of nanopillars comprising a second metal, each pair of nanopillars comprising a first nanopillar and a second nanopillar spaced-apart from the first nanopillar by a nanopillar gap, wherein a bottom surface of the first nanopillar is physically and electrically connected to the first nanoelectrode, and a bottom surface of the second nanopillar is physically and electrically connected to the second nanoelectrode, and wherein the first and second nanopillars each comprise posts projecting substantially vertically through the resist or dielectric layer such that only a top surface of each nanopillar is uncovered by the resist or dielectric layer.

In various embodiments, the top surface of each nanopillar is: (a) protruding beyond a top surface of the resist or dielectric layer; (b) flush with the top surface of the resist or dielectric layer; or (c) recessed below the top surface of the resist or dielectric layer.

In various embodiments, the structure further comprises a bridge molecule having a first end and a second end, the first end of the bridge molecule bonded to the first nanopillar and the second end of the bridge molecule bonded to the second nanopillar, bridging the nanopillar gap.

In various embodiments, the first metal comprises Al, Cu, Ru, Pt, Pd, or Au, and the second metal comprises Ru, Pt, Pd, or Au. In various embodiments, the first metal comprises Al and the second metal comprises Ru.

In various embodiments, the top surface of at least one nanopillar in the pair of nanopillars comprises a mushroom protrusion extending the nanopillar horizontally over a portion of a top surface of the resist or dielectric layer.

In various embodiments, only one nanopillar in the pair of nanopillars further comprises a horizontal portion extending across a portion of a top surface of the resist or dielectric layer and toward the other nanopillar in the pair of nanopillars.

In various embodiments, at least one nanopillar in the pair of nanopillars comprises a vertically tapered nanopillar, and wherein a bottom portion of the vertically tapered nanopillar is larger in diameter than a top portion of the vertically tapered nanopillar.

In various embodiments, both nanopillars in the pair of nanopillars comprise vertically tapered nanopillars.

In various embodiments, a method comprises: depositing a pair of nanoelectrodes on a substrate, the pair of nanoelectrodes comprising a first metal and including a first nanoelectrode and a second nanoelectrode spaced-apart from the first electrode by a nanogap; applying a resist coating to form a resist layer over the pair of nanoelectrodes and the nanogap, the resist layer having a horizontal exposed top surface; patterning a pair of open holes vertically through the resist layer, the patterning comprising one hole per nanoelectrode, each hole beginning with an exposed portion of the nanoelectrode and extending vertically from the nanoelectrode through the resist layer, ending in an opening at the horizontal exposed top surface of the resist layer; and depositing a second metal into each hole to form a pair of nanopillars, each nanopillar formed in the shape of the hole, the nanopillar having a bottom portion in physical and electrical contact with the nanoelectrode and an exposed top surface near, at, or protruding above the horizontal exposed top surface of the resist layer.

In various embodiments, the substrate comprises a Si layer and a SiO₂ insulative layer onto which the nanoelectrodes are deposited.

In various embodiments, the method further comprises the step of planarizing the horizontal exposed top surface of the resist layer after the step of depositing the second metal such that the exposed top surface of each nanopillar is flush with the horizontal exposed top surface of the resist layer.

In various embodiments, the exposed top surface of each nanopillar comprises a circular shape.

In various embodiments, the method further comprises the step of bonding a bridge molecule between the pair of nanopillars, such that a first end of the bridge molecule is bonded to one nanopillar and a second end of the bridge molecule is bonded to the other nanopillar in the pair of nanopillars.

In various embodiments, the depositing of second metal is continued for a time sufficient to produce a mushroom protrusion on the top surface of each nanopillar extending vertically above and horizontally across a portion of the horizontal exposed top surface of the resist layer.

In various embodiments, the method further comprises, after the step of depositing the second metal, the step of direction-guided electrodeposition of additional second metal on one nanopillar creating a horizontally disposed portion on the one nanopillar extending across the horizontal exposed top surface of the resist layer in a direction toward the other nanopillar in the pair of nanopillars.

In various embodiments, the method further comprises, after the step of patterning the pair of open holes, the step of adding resist coating into a top portion of each of the patterned open holes to reduce the size of each opening of each hole.

In various embodiments, the method further comprises, after the step of depositing the second metal, the additional steps of: dissolving away the resist layer to leave exposed nanopillars; reducing the diameter of and optionally vertically tapering each nanopillar by an etching process; casting a new resist layer to entirely cover the nanopillars; planarizing the resist layer such that a top surface of each nanopillar is flush with a top surface of the resist layer; dissolving away each nanopillar to leave behind a hole; depositing a material into each hole to create nanopillars physically and electrically attached to the nanoelectrodes.

In various embodiments, the first metal comprises Al, Cu, Ru, Pt, Pd or Au, the second metal comprises Cu or Ni, and the material comprises Ru, Pt, Pd or Au.

In various embodiments, a method comprises: depositing a pair of nanoelectrodes on a substrate, the pair of nanoelectrodes comprising a metal or semiconducting material and including a first nanoelectrode and a second nanoelectrode spaced-apart from the first nanoelectrode by a first nanogap; choosing a second nanogap having distance less than the first nanogap; determining an electroless deposition duration time required to narrow the first nanogap down to the second nanogap by interpolating the second nanogap on an x/y plot of nanogap distance versus electroless deposition duration time; and preforming electroless deposition of a metal or noble metal on the nanoelectrodes for the electroless deposition duration time thus determined, producing the second nanogap between the nanoelectrodes.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the following drawing figures:

FIG. 1 illustrates with cross-sectional views a method to form nanoscale pillars (“nanopillars”) on electrodes within an array of electrodes in accordance with various embodiments of the present disclosure;

FIG. 2 illustrates a top view of an array of electrode pairs comprising an imprint mask that was used to direct formation of nanopillars within holes prepared in the imprint mask in accordance with various embodiments of the present disclosure;

FIG. 3 illustrates the overall concept of “tunable nanopillars” through exemplary embodiments having progressively narrower electrode gaps obtained by specific depositing in accordance with various embodiments of the present disclosure;

FIG. 4 illustrates a method of narrowing nanopillar diameter at only the top portion of each nanopillar in accordance with various embodiments of the present disclosure;

FIG. 5 illustrates various examples of tapered nanopillars having protruding, recessed or flushed tips relative to a dielectric layer surface in accordance with various embodiments of the present disclosure;

FIG. 6 illustrates a method of forming nanopillars through use of sacrificial metal nanopillars in accordance with various embodiments of the present disclosure;

FIG. 7 illustrates a nanopillar array fabrication process method on CMOS-compatible Cu films in accordance with various embodiments of the present disclosure;

FIG. 8 illustrates nanofabrication steps for producing nanopillar arrays on circuit chip devices, using either physical vapor deposition (sputtering or evaporation), or electrodeposition or electroless deposition of Au in accordance with various embodiments of the present disclosure;

FIG. 9 is a drawing of an SEM micrograph, taken in tilted view, showing sputter-deposited and lift-off processed 30 nm diameter gold (Au) nanopillar top on nanopillar structure on CMOS-compatible Cu metallization in accordance with various embodiments of the present disclosure;

FIG. 10 is a drawing of an SEM micrograph of an array of nanopatterned Au nanopillar top circles exposed flush on a planarized 50 nm tall Sift dielectric layer in accordance with various embodiments of the present disclosure;

FIG. 11 illustrates top views of various nanoelectrode geometries in accordance with various embodiments of the present disclosure;

FIG. 12 illustrates a method of nanoelectrode gap control by electrochemical deposition in accordance with various embodiments of the present disclosure;

FIG. 13 provides drawings of various SEM micrographs of a rectangular Au electrode pair showing a closing electrode gap dimension versus duration of electroless Au deposition at 90° C., pH 8, in accordance with various embodiments of the present disclosure; and

FIG. 14 is an x/y plot of the data from FIG. 13, electrode nanogap dimension vs duration of electroless Au deposition time in accordance with various embodiments of the present disclosure. The plot allows interpolation of the time needed to produce a desired nanogap distance between electrodes.

It is to be understood that the drawings are for purposes of illustrating the concepts of various embodiments disclosed herein and are not necessarily drawn to scale.

### DETAILED DESCRIPTION

The detailed description of exemplary embodiments refers to the accompanying drawings, which show exemplary embodiments by way of illustration and best mode. While these exemplary embodiments are described in enough detail to enable those skilled in the art to practice the invention, other embodiments may be realized, and logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments of the present disclosure, new lithographic methods and nanoscale structures are provided that find use in molecular electronics sensors, such as molecular sensors for nucleotide sequencing. In various embodiments, the concept of tunable nanopillars is introduced and described, wherein nanoscale pillars called “nanopillars,” extending substantially vertically from electrode surfaces, are customizable in shape and size, and in some embodiments are used to provide specific gap distances between adjacent electrodes that comprise such nanopillars. In various embodiments, tunable nanopillars provide suitable gap distances between pillars for bridging a biomolecular across the gap.

In various embodiments, tunable nanopillars and other nanoscale structures obtained by various lithographic methods find use in molecular electronic sensors. In particular, the structures and methods herein find use in the sensors described in U.S. Pat. No. 10,508,296 and U.S. patent application Ser. No. 16/015,049, filed Jun. 21, 2018, both of which are incorporated herein by reference in their entireties for all purposes.

### Definitions and Interpretations

As used herein, the directional terms “top,” bottom,” “up,” “down,” “horizontal,” “vertical,” etc., are relative to a generally flat substrate onto which various components and layers are disposed. Certainly, a substrate could be inverted and turned around in various ways, even during lithographic processes, therefore it is helpful to standardize these relative directions to a generally flat substrate, like a semiconductor chip, in the orientation where it is situated flat like a tile sitting on a table. The substrate, being generally flat like a tile, has a horizontal top surface onto which materials are disposed. Certain structures, like electrodes, may be disposed on the substrate, wherein the electrodes have a length and a width in the horizontal plane defined by the top surface of the substrate, having an exposed top surface opposite the substrate, and projecting upwards from the substrate by a certain electrode thickness. Nanopillars, defined below, are described as projecting in a substantially vertical direction from a substantially planar electrode surface. Given these directional considerations, the nanopillars can be said to project orthogonally or vertically from the horizontal plane of the substrate. The projection is described as “up,” since the lithography is performed on the top surface of the electrodes. Further, as a layer of material, like a resist, may be coated onto the substrate, the layer will necessarily include both a bottom surface situated against the underlying structure it was applied to, and a top exposed surface that is substantially horizontal and generally parallel to the horizontal plane of the underlying substrate.

As used herein, the term “electrode” takes on its ordinary meaning of a conductive or semiconducting element found in an electronic circuit, configured to act as an efficient source or drain of electrons or other charge carriers. In various embodiments, electrodes herein comprise metallic materials or semiconducting materials, such as might be found in electronics, and may be of any shape, such as rectangular, spearhead, pointed tip, rounded with a pointed tip, etc. In various embodiments, electrodes herein may comprise aluminum (Al), copper (Cu), ruthenium (Ru), platinum (Pt), palladium (Pd) or gold (Au), recognizing that Au would likely not be the metal of choice for electrodes made in a chip foundry. Electrodes herein are formed on substrates, such as by lithography, and may be organized in arrays on top of a CMOS chip, such as a sensor pixel array device, either directly manufactured on top of a CMOS device, or manufactured on a separate substrate that can be electrically mated to such a device using through-silicon-via (TSV) connectors.

In various embodiments, electrodes herein are arrayed in pairs of nanoscale electrodes (called “nanoelectrodes”), with each pair of electrodes comprising two electrodes spaced-apart by a nanoscale distance referred to as a “nanogap.” The nanogap is the distance between the closest edges of the two electrodes, recognizing that electrodes may be elongated in shape, such as rectangular. In various embodiments herein, nanoelectrodes are of nanoscale dimensions, For example, rectangular nanoelectrodes may measure from about 1 nm×100 nm in length by about 0.5 nm to about 50 nm in width. In various embodiments, an electrode pair herein comprises one (+) and one (−) electrode, or one source and one drain electrode. For simplicity, the two electrodes in a pair of electrodes may be referred to as a first and a second electrode. In various embodiments, an electrode may comprise a gate electrode, which can be disposed between source and drain electrodes for applying a bias to a circuit comprising the pair of electrodes. In various embodiments, microscale electrodes may be disposed in contact with a nanoelectrode, providing an electrical conduit to a ganged arrangement. A pair of microelectrodes would not typically participate in the bonding of biomolecules, and would be on the outer opposite sides of a pair of nanoelectrodes, opposite the nanogap.

As used herein, the term “nanopillar” refers to a nanoscale structure formed on an electrode. In various embodiments, a nanopillar comprises a substantially vertically projecting post, rod or pillar-like shape, emanating from a larger portion of an electrode, such as a horizontally planar portion of an underlying electrode. In other words, a nanopillar may be seen as a vertical extension of an otherwise horizontally disposed electrode, wherein that extension is customized in shape and size for a particular function. In various embodiments, a nanopillar can be described as having a central axis that is orthogonal or nearly orthogonal to a horizontal plane defined by the generally flat electrode and substrate surfaces as per above. In some instances, nanopillars herein may have unique shapes instead of cylinders or rectangular posts, such as “milk bottle” shapes, wherein a bulbous base portion projects and narrows vertically into a narrow top portion. In various instances, the very top of a nanopillar may be reduced in size so that the number of biomolecules likely to bond to the nanopillar is reduced in probability to just one or at most just a few. The top of a nanopillar may be flat and circular, such as when the nanopillar is cylindrical and is planarized, or the top may be rounded into a semicircular shape. Nanopillars may also have a pentagonal, square or triangular cross section or any other shaped cross section rather than a circle. Nanopillars may comprise a conducting or semiconducting material, which may be the same or different from the material used in the electrode onto which the nanopillar is disposed. In various embodiments, nanopillars comprise ruthenium (Ru), platinum (Pt), palladium (Pd), gold (Au), or if sacrificial, copper (Cu) or nickel (Ni), again recognizing that gold (Au) might not be the metal of choice for nanopillars made in a semiconductor foundry. This limitation is of course not present if nanopillars are made by electroless plating post foundry. In various embodiments, the material for a nanopillar is chosen for its ability to bind a material binding domain configured on the end of a biomolecule, such as a functional group that can form a bond to a metal. In various embodiments, nanopillars of a second metal provide vertical extensions to the electrodes comprising a first metal to which the nanopillars are physically and electrically attached. In various arrangements, a pair of nanopillars may be disposed on a pair of electrodes, one nanopillar per electrode, wherein the electrodes are spaced-apart by a nanogap and the nanopillars are spaced-apart by a nanopillar gap. Depending on how close to the nanogap the nanopillars are disposed, the nanopillar gap may be just wider than the nanogap. At times, either the nanogap or the nanopillar gap may be referred to as an “electrode gap,” recognizing that either the pair of electrodes, or the pair or nanopillars, or one of each may be involved in the bonding of a bridge molecule spanning the electrode gap.

As used herein, the term “tunable” refers to the ability to provide a discrete shape or dimension to a structure. Thus, the phrase “tunable nanopillar” refers to a nanopillar that can be adjusted to a particular shape and/or size to address a certain need. In other words, tunable herein is the same as dimensionally adjustable. For example, a pair of spaced-apart tunable nanopillars may be adjusted in shape and/or size to optimize the distance between them and the probability that a biomolecule of particular size and chemical type can bridge the gap between them.

As used herein, the term “bridge molecule” or “biomolecular bridge” or “biomolecule” indicates an organic molecule generally comprising a linear chemical structure, such as a synthetic semisynthetic, natural or genetically engineers linear polymer, having at least some electrical conductivity. Such molecules are intended for use with the structures and devices disclosed herein, wherein a bridge molecule “bridges” across an electrode gap disposed between spaced-apart electrodes to close an otherwise open electrical circuit. As such, a bridge molecule herein will be a molecule having a length that substantially exceeds its steric width, wherein the length may be from about 5 nm to about 100 nm and the width only about 1 nm to about 5 nm. A bridge molecule for use in molecular sensors comprise a first end and a second end, wherein the first end is configured to bond to a first electrode or first nanopillar and the second end is configured to bond to a second electrode or second nanopillar. In various embodiments, a bridge molecule may comprise an oligonucleotide or a polypeptide, proteins or fragments thereof (e.g., an α-helix portion of a protein natural or engineered, or an antibody or portion of an antibody), nanotubes, graphene nanoribbons, other fused polycyclic aromatic substances, synthetic linear polymers such as 2,5-(poly)thiophene, etc., with the first and second ends of the molecule configured with material binding domains comprising an amino acid, amino acid sequence, of functional groups such as —SH groups or other sulfur-containing functional groups.

In various embodiments, biomolecules configured for use a bridge molecules in molecular sensors are functionalized at both a first end and second end to promote bonding of each end of the molecule to metal. In various embodiments, a “functional length” of a bridge molecule includes the functionality configured for metal electrode or nanopillar binding, such that the spacing between nanopillars in a pair of nanopillars, or the spacing between electrodes in a pair of electrodes, can be matched to the functional length of the biomolecule intended to bridge between the pair of nanopillars or electrodes, such that the bridging is promoted. In various embodiments, a bridge molecule may bridge between a nanopillar and an electrode, rather than between electrodes or between nanopillars. In various embodiments, a bridge molecule may also be configured with functionality for binding a probe molecule to the bridge molecule, such as near the midpoint of the length of the bridge molecule. Such functionality may be one partner for click-chemistry, with the other partner being present on the probe molecule.

As used herein, the term “sensor complex” refers to a combination of bridge molecule and probe molecule, wherein the probe molecule is conjugated to the bridge molecule somewhere between the first and second ends of the bridge molecule. In various examples, a sensor complex may comprise a polymerase or other processive enzyme conjugated to a biomolecular bridge molecule such as an oligonucleotide or polypeptide. In the construction of a molecular electronics sensor, a bridge molecule may first be bonded across a pair of nanoelectrodes or nanopillars, or between one of each, and then a probe molecule may be conjugated to the bridge molecule. In other embodiments, a probe molecule may first be conjugated to a bridge molecule to form a sensor complex, and then the sensor complex is bonded between nanoelectrodes or nanopillars, or one of each to form a closed circuit.

### General Embodiments and Considerations

For molecular electronic sensors comprising a biomolecule acting as a conductive circuit element, a pair of spaced-apart electrodes (optionally with a third electrode configured as a gate electrode) is required. For label-free molecular sensors configured for genome sequencing without complicated fluorescence imaging, molecular sensors comprising a DNA molecule as a molecular bridge between spaced-apart electrodes is one way of enabling such analysis. It has been previously found possible to attach a single polymerase enzyme molecule, or other type of binding probe, to a DNA bridge molecule or other bridging biomolecule such as a polypeptide by using functionalities and ligands such as biotin-streptavidin, antibody-antigen, or peptide complexes. Such molecular sensors comprising a sensor complex further comprising a biomolecular bridge molecule spanning an electrode gap and a binding probe bonded thereto are taught in the '296 patent and the '049 application, amongst other disclosures of the same assignee.

In various embodiments, the electrodes taught in both the '296 patent and '049 application may have widths from about 20 nm to about 50 nm, and are made, for example, by deposition of material on substrates by nanofabrication techniques such as like e-beam lithography, EUV lithography and nanoimprint lithography. In stark contrast, the diameter of a DNA oligonucleotide usable as a molecular bridge across spaced-apart electrodes in a pair of electrodes is only about 1 nm. For optimal performance of molecular sensors, such as those disclosed in the '296 patent and '049 application, only a single bridge molecule should span each electrode gap, i.e., one bridge molecule per electrode pair. In producing a DNA bridge across spaced-apart electrodes, a single DNA bridge is the most preferred, although having a few parallel DNA bridges across a single electrode gap may still be usable. On a 20 nm to 50 nm wide electrode strip, many ˜1 nm diameter DNA oligonucleotides can attach, which can cause complicated signal mix-ups in a molecular sensor, which in turn can make discerning individual nucleotide interactions with a molecular sensor complex, nucleotide identification, and ultimately, a nucleotide sequence, very difficult. Therefore, if the size of an exposed portion of an electrode can be reduced to as small an area as possible (e.g., less than about 10 nm, and even less than about 5 nm), single molecule bridges or at most just a few molecular bridges will tend to form on each electrode pair. In various embodiments, the area on an electrode for biomolecular bridge binding should be reduced to less than about 10 nm, or less than about 5 nm, in diameter.

Another aspect of electrode structure in a molecular sensor comprising pairs of spaced-apart electrodes is the nanoscale gap distance (“nanogap”) provided between the two adjacent electrode tips in any pair of spaced-apart electrodes. Depending on specific bridge molecule types and lengths (e.g., DNA oligonucleotides, polypeptides, antibody fragments, etc.), which may be quite variable, the nanogap distance has to be adjusted so that the gap dimension is comparable to the biomolecule length. Therefore, it is highly desirable if the nanogap distance can be adjustable through various lithographic techniques in order to accommodate various bridge molecules.

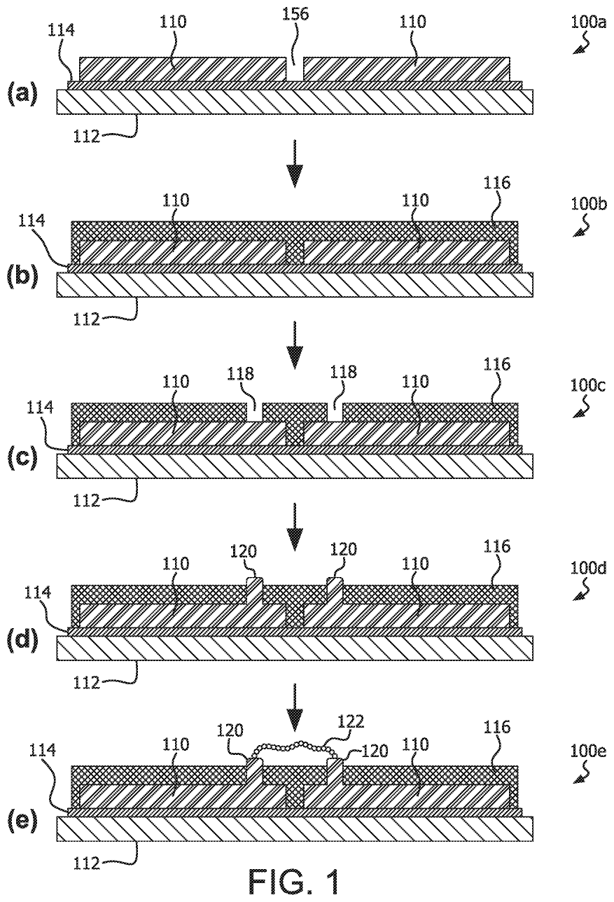

In various embodiments of the present disclosure, methods for manufacturing very precise and reliable electrode structures comprising a tunable (i.e., size-confineable) nanopillar diameter as well as a tunable nanogap dimension are disclosed. Various embodiments of the present structures and methods are set forth in FIGS. 1-14, along with experimental data supporting a reduction-to-practice. Such tunable electrode structures are also manufacturable for large scale production and are amenable to manufacturing large arrays of electrodes.

### Illustrative Embodiments

Referring now to FIG. 1, a method for producing nanopillars on electrodes is described. The structures 100a through 100e, represent various sequential steps in the method, and are illustrated in cross section for clarity. In FIG. 1, structure 100a comprises a pair of electrodes 110 deposited on a substrate 112 and separated by a nanogap 156, wherein the substrate optionally comprises an oxide or other insulative layer 114. For example, the substrate 112 may comprise Si, whereas the insulative layer 114 may comprise SiO₂. In various embodiments, the electrodes 110 may comprise a metal such as Al, Cu, Ru, Pt, Pd or Au, or another conducting or semiconducting material. As mentioned above, the bare electrodes 110, deposited in arrays on the substrate, may not be suitable for bridging just a single biomolecule, or at most just a few biomolecules, across nanogap 156. In some instances, electrodes 110 have too much exposed surface area, and may be prone to binding a multitude of biomolecules, both bridging across the nanogap and binding to the same electrode in a loop. To reduce electrode bonding area and mitigating these potential problems, nanopillars are constructed.

As shown in FIG. 1, structure 100b, a resist coating 116 is applied over the entire structure, covering both electrodes 110 in the pair of electrodes and filling in the nanogap 156 between them. The resist layer remains with an exposed horizontal top surface opposite the side covering the electrodes and nanogap. The resist may comprise a positive resist, such as polymethylmethacrylate (PMMA), or a negative resist, such as hydrogen silsesquioxane (HSQ), or the epoxy resin based SU-8 (from MicroChem, Newton, Mass.). Other materials are usable, including SiO₂ or other dielectric material. The thickness of the resist is determined at least in part by the height desired for the finished nanopillars, recognizing that surfaces can be planarized to not only level heights but to overall shorten heights. In FIG. 1 structure 100c, the resist coating 116 is then patterned with an array of holes 118 by e-beam lithography or nano-imprinting. The patterning may be conducted across 10's of thousands of electrode pairs in large arrays on chips. The holes 118 are imprinted in rows such that there is only one hole 118 per electrode 110, and each hole 118 is made near the end of the electrode that is adjacent to its companion electrode in the pair of electrodes. Each patterned hole is open, beginning with an exposed portion of the electrode being the bottom of the hole, and vertically extending through the thickness of the resist layer ending at an opening at the top surface of the resist layer. The distance between paired holes 118 on a pair of electrodes determines the gap distance for a bridging biomolecule. In other words, a biomolecule will bind across the finished nanopillars rather than across the original nanogap 156 between the electrodes. In various embodiments, each hole is from about 3 nm to about 30 nm in diameter, and about 3 nm to about 100 nm in height. Each pair of holes 118 are spaced apart in relation to the length of the biomolecule that will be used to bridge nanopillars. For example, an oligonucleotide or polypeptide having a first end and a second end for bridging across a pair of nanopillars may have a functional length (i.e., including material binding regions at both first and second ends) that will be approximated in the resist patterning.

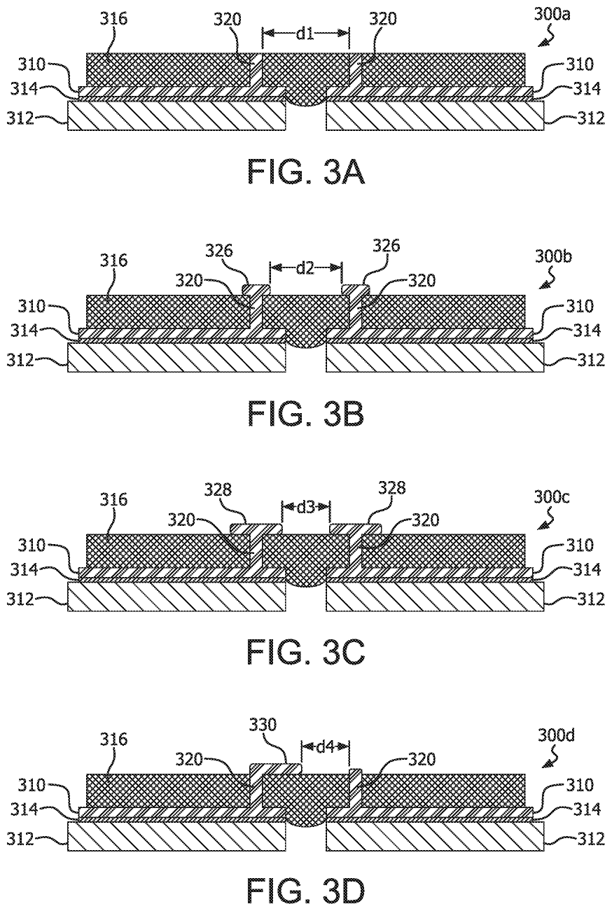

As shown now in FIG. 1 structure 100d, protruding nanopillars 120, such as comprising Ru, Pt, Pd or Au, are now grown in the patterned holes, with the initial depositing directed in contact with the exposed portions of the electrodes at the bottom of each hole and with continued depositing to grow the vertical pillars substantially in the shape of the holes. The depositing may be to a height below the height of the resist, to a height at the level of the resist, or to a height that exceeds the level of the resist as illustrated in this particular example. Depositing of metal or alloy may be accomplished by electroless or electroplating deposition, or by a sputtering and lift-off process. The resulting structure may be planarized if needed so as to level the height of the nanopillars to the height of the resist layer and/or to shorten the height of the array of nanopillars and resist. Such a planarized structure is illustrated as 300a in FIG. 3, (discussed below).

FIG. 1 structure 100e shows how only one biomolecule 122, or at most just a few, will bridge across a pair of nanopillars, partly because the small exposed tip of each nanopillar (e.g., measuring only about 3-10 nm in diameter) can only accommodate a single metal-biomolecule bond, such as a thiol-Au bond between the pillar and a thiol functional group at the end of the biomolecule. In various embodiments, the nanopillar deposits 120 comprise the same metal as the underlying electrodes 110 to ensure strong mechanical and electrical connection between each nanopillar and the electrode surface directly beneath and in contact with the nanopillar. In other words, when the material of the electrode 110 and the nanopillar 120 are the same, the nanopillar is in all respects a vertically projecting extension of the electrode. With the method illustrated in FIG. 1, massively parallel and size-reduced “single-pair islands” are produced on an electrode array via masked electrode deposition.

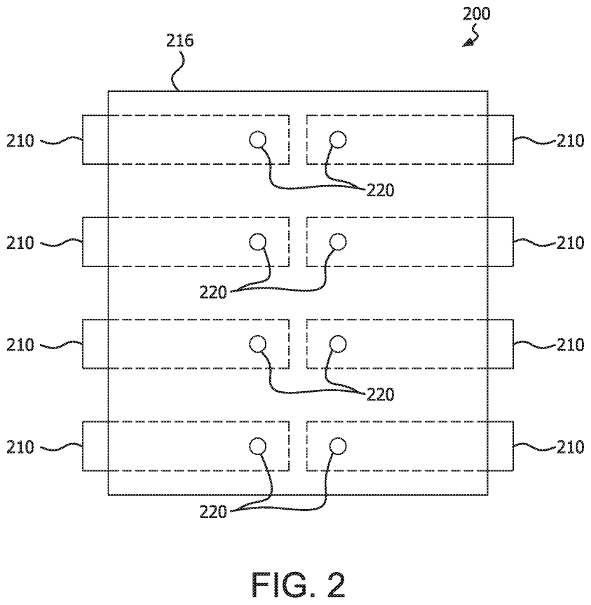

With reference now to FIG. 2, array 200, resulting from the method of FIG. 1, is shown in top view (without the biomolecular bridges). Array 200 comprises an array of electrode pairs 210 covered with resist layer 216 and reduced nanopillar regions 220 exposed for bonding of biomolecular bridge molecules. The method of FIG. 1 provides reduced-area regions 220 suitable for attaching a single or only a few biomolecules for bridge formation. In various embodiments, these reduced-area regions 220, i.e., the exposed tops of the nanopillars, are from about 3 nm to about 10 nm in diameter, by use of a nano-imprint mask 216. In various embodiments, Ru, Pt, Pd, or Au, or other noble metal or alloy, may be deposited in the patterned holes to create the vertical nanopillar pair for each pair of electrodes in an array of electrode pairs. The top view of array 200 also shows how the resist mask 216 covers all of the remaining areas of the electrodes 210 to block any spurious bonding of biomolecules. In various embodiments, a fluidic cell is built around the array such that buffer solution containing the biomolecules for bridging the nanopillars only reaches the masked area and not the exposed ends of the electrodes that are used for electrical lead connection.

FIG. 3 illustrates the general concept of “tunable nanopillars,” by showing examples how controlled depositing can “tune,” i.e., dimensionally adjust, the distance between nanopillar structures, so as to promote bridging of biomolecules having a particular length. In examples (a), (b), (c), and (d), both the nanopillars and the underlying electrodes may comprise Ru, Pt, Pd or Au, or the nanopillars and underlying electrodes may comprise different metals.

With reference now to FIG. 3A, structure 300a comprises a pair of electrodes 310 and associated nanopillars 320, such as obtained per the method of FIG. 1. Structure 300a is obtained by planarizing structure 100d from FIG. 1 so as to level the heights of the pair of nanopillars 320 with the height of the resist layer 316. Structure 300a comprises nanopillars 320 separated by a distance d1, which in various embodiments may measure about 20 nm. This gap d1 would thus be suitable for bridging a single molecule or at most a few molecules having a functional length of about 20 nm (including material binding portions provided at each of the two ends of the molecule) across distance d1 between nanopillars 320. The presence of resist or SiO₂ dielectric 316 between the nanopillars 320 prevents sagging of the biomolecular bridge molecule into what was the original nanogap between electrodes, where the molecule could interact with the electrodes causing a type of short circuiting of the sensor. In various embodiments, structure 300a can be exposed to a solution of biomolecules of functional length about 20 nm, upon which a single or at most just a few may bridge across the 20 nm distance d1 between nanopillars 320. If the distance d1 wasn't suitable, or is no longer suitable, such as if the sensor is to be reconfigured for use with other bridge molecules having functional length less than d1, then the methods described below may be employed for tuning the distance between tunable nanopillars.

With reference now to FIG. 3B, structure 300b comprises nanopillars 320 with mushroom protrusions 326 deposited on the vertical nanopillars 320 in structure 300a, extending the nanopillar both vertically above the surface of the resist layer 316 and also horizontally over a portion of the top surface of the resist layer 316. In various embodiments, extended depositing of Ru, Pt, Pd or Au or other metal or alloy for a time sufficient, by electroless or electroplating deposition, or by a sputtering and lift-off process, forms the mushroom protrusion 326 on the nanopillars 320, shortening the distance between nanopillars from d1 to d2. In various embodiments, d2 may be about 16 nm. Certainly, deposition may be continued for longer periods of time to further reduce the distance d2 between protrusions 326, but recognizing that the exposed surfaces of 326 correspondingly increase in size, risking again the possibility that multiple biomolecules may bond to the protrusion 326.

So, for example, and as illustrated in FIG. 3C, extended deposition to create the mushroom protrusions 328 in structure 300c may be for a time sufficient to tune the desired gap d3 to a desired distance, but not for such a long period of time to create overly large mushroom protrusions 328 that would otherwise promote multiple bridge molecule binding. The extended deposition from 300b to 300c may in various embodiments provide a gap d3 of about 12 nm or less. In summary, the method of extended deposition, transitioning from the basic nanopillars in 300a to the smaller mushroom protrusions 326 in 300b and then further to the larger mushroom protrusions 328 in 300c, shorten the distance between nanopillar structures from d1 to d2 to d3, or from about 20 nm to about 16 nm, and finally to about 12 nm or less.

FIG. 3D illustrates a method of direction-guided electrodeposition to tune the gap between tunable nanopillars by extending only one nanopillar with a horizontal portion extending toward the adjacent nanopillar. The structure 300d may be obtained from structure 300a by extending only one of the nanopillars in a direction toward the other nanopillar in the pair by polarity controlled electrodeposition. As shown in structure 300d, one of the nanopillars 320 in the pair of nanopillars further comprises a horizontal portion 330 extending across the top surface of the resist layer 316 toward the other nanopillar 320 in the pair of nanopillars. In this way, the original spacing d1 between nanopillars 320 in 300a is tuned to a new distance d4, which in various embodiments may be about 12 nm or less. So, in some regards, direction-guided electrodeposition, giving rise to structure 300d, can be an alternative process to extended deposition on both nanopillar tops that gives rise to structure 300c, recognizing that in various embodiments, the two independent process may give rise to the same or different distance between nanopillars (i.e., d3 versus d4). In other embodiments, polarity can be reversed and the other nanopillar in the pair of nanopillars 320 illustrated can also be extended in the horizonal direction toward the nanopillar already having the horizontally extending portion. In this way, both nanopillars can comprise the horizonal extension, extending toward one another.

As described by way of the examples in FIG. 3, nanopillars 320 are ultimately tunable nanopillars, in that the gap between exposed nanopillar tops (e.g., between pair of 326 protrusions in 300b, between pair of 328 protrusions in 300c, or between extension 330 and 320 in 300d) is ultimately tunable, wherein the distance d1, d2, d3, d4 is controlled by electroless deposition or direction-guided electrodeposition.

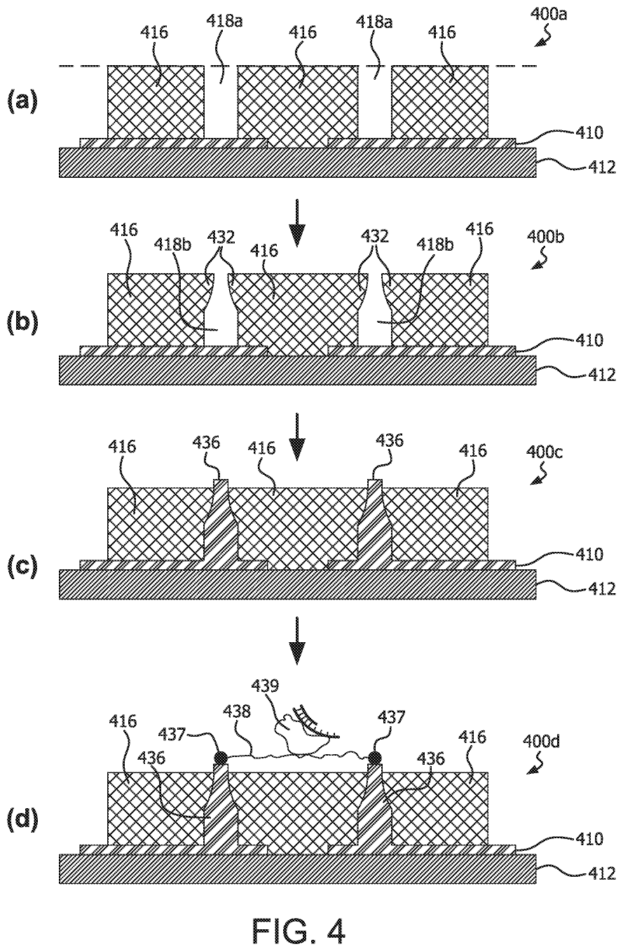

FIG. 4 sets forth various embodiments of a method for reducing the size of the exposed top surfaces of nanopillars, such that the resulting structure is optimized for single bridge molecule binding across paired nanopillars. The method exemplified in FIG. 4 begins with structure 100c of FIG. 1, or its equivalent, which is then planarized (as per the dashed horizonal line in 400a) to provide beginning structure 400a in FIG. 4. The structure 400a comprises nanoimprinted or e-beam lithographed holes 418a in the resist coating 416, e.g., PMMA or SiO₂, wherein one hole 418a is patterned directly over only one electrode 410, as per the method of FIG. 1, along with the underlying electrodes 410 that were previously deposited on substrate 412. In various embodiments, the holes 418a may be close to cylindrical in shape, having uniform diameters of about 25 nm. The next step in the method for reducing the size of the exposed top surfaces of the nanopillars is to reduce the diameter of just a top portion of each hole 418a, to less than about 10 nm, or in certain embodiments, to less than about 5 nm, by closing in the circumference of the resist layer surrounding each hole with additional resist material 432, as depicted in structure 400b. This is accomplished by any one or combination of resist spin coating, oblique angle or vertical low-pressure sputtering, evaporation of silica or other dielectric layer, or other nano-processing methods, with optional planarizing by reactive ion etching (RIE), etc. For example, the vertical holes 418a can be made tapered by spin-coating of PMMA or HSQ (to be later converted to SiO₂), or sputter-coated with oxide dielectric layer to selectively reduce the diameter of the top region of each hole. In various embodiments, material can be added toward the top of the holes 418a, or material removed from the bottom of the holes 418a, to achieve these uniquely shaped holes 418b. As illustrated in structure 400c, resulting from this step are holes 418b comprising a contoured “milk bottle shape” rather than straight cylinders.

With continued reference to FIG. 4, the next step in the method, illustrated by a transition from structure 400b to 400c, comprises depositing metal or alloy material, such as Ru, Pt, Pd, or Au, into each hole 418b to produce nanopillars 436 that are physically and electrically connected to the underlying electrodes 410. This step can be the same as previously described for the method in FIG. 1, wherein each hole 418b is filled in by metal depositing using methods such as electroless plating or sputter deposited and lift-off processed, etc. The result is shown by structure 400c wherein nanopillars 436 are formed, having a contoured shape wherein a top portion is narrower than a bottom portion. So, for example, the nanopillars 436 may have a diameter near the top of the nanopillar of less than about 10 nm, or less than about 5 nm, and a diameter at the bottom of the nanopillar, where the nanopillar merges to the underlying electrode 410, of about 25 nm. In effect, the uniquely shaped nanopillars 436 are vertically projecting extensions of the underlying electrodes 410, finishing at very small exposed diameters at the previously planarized level of the resist layer 416.

With continuing reference to FIG. 4, the last step in the method thus illustrated is to bridge a biomolecule 438 between the pair of nanopillars 436 and to conjugate a binding probe 439 to the bridge molecule. These steps can be reversed in order, wherein a probe complex comprising the binding probe 439 and bridge molecule 438 is bridged between the pair of nanopillars 436. In various embodiments, the conjugations 437 shown between each end of the bridge molecule 438 and each of the contoured nanopillars 436 may comprise thiol-Au bonding, or in general, any covalent or non-covalent linkage or association between the metal conducting, semiconducting, or alloy nanopillar 436 and a material binding domain configured on each end of the biomolecule 438. In various embodiments, the material binding domain configured on each end of the bridge molecule 438 may comprise individual amino acids or short polypeptides capable of bonding to metals. In other embodiments, the conjugations 437 may comprise any type of “click chemistry” between a functional group on the nanopillar and a functional group configured on the end of the biomolecule 438. In various embodiments, the molecular bridge 438 may comprise a single- or double-stranded DNA oligonucleotide, in some instances diazonium-enhanced, or a polypeptide comprising an α-helix portion, or an entire α-helix such as devised by genetically engineering sequences of amino acids that naturally express α-helix structure.

In various embodiments, the binding probe 439 in structure 400d may comprise a polymerase or other processive enzyme. An array of such 400d subunits, such as disposed on a CMOS chip, act as a solid state molecular electronics sensor. In various embodiments, the structure 400d is part of an array of sensors used in nucleotide sequencing, wherein the array may be enclosed in a fluid chamber to facilitate delivery of solutions of dNTPs. The strip of material shown interacting with binding probe 439 may comprise a single-stranded DNA template being processed by the processive enzyme 439. The interaction of dNTPs with the binding probe 439 may cause a change in current pulse or other signals that can be detected and related to a nucleotide sequence. These methods are amply disclosed in the '296 patent and the '049 application, reference above and incorporated herein by reference. Suffice it to say that the structure 400d in FIG. 4 can be the foundation of a molecular electronics sensor used for nucleotide sequencing as describing in the '296 patent and the '049 application.

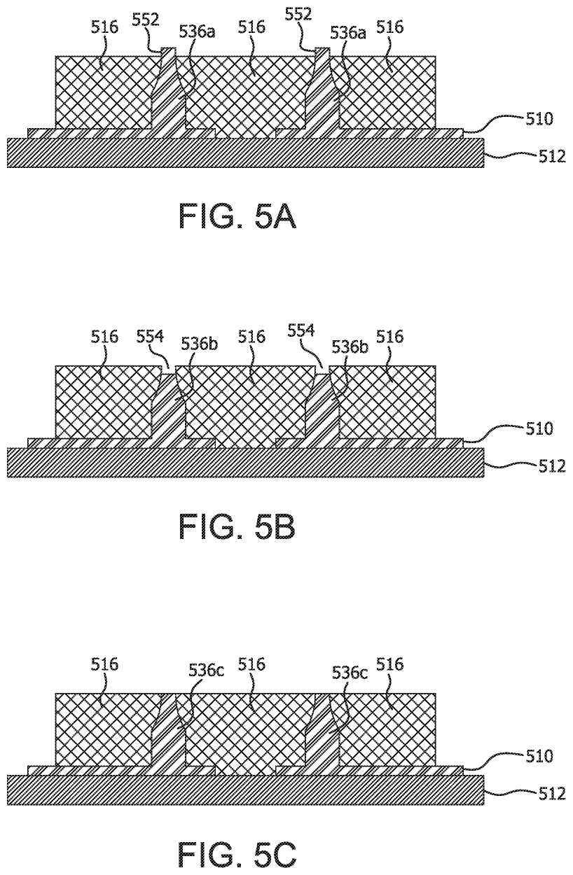

FIG. 5 illustrates optional further manipulations of the structure 400c in FIG. 4, or its equivalents. As shown in FIG. 5A, the depositing of metal into the contour shaped holes may produce nanopillars 536a having a portion 552 extending above the surface of the resist layer 516. In this case, the structure comprises electrodes 510 on a substrate 512 and contoured nanopillars 536a protruding beyond the horizontal surface of resist layer 516 by portions 552. In other embodiments, and as shown in FIG. 5B, depositing of the Ru, Pt, Pd, or Au, or other material such as another noble metal, into the holes may be been terminated early such that the resulting contoured nanopillars 536b end short of the top level surface of the resist layer 516, creating a recess 554 above each nanopillar 536b. In yet a third variation, and as illustrated in FIG. 5C, the structure (regardless if the nanopillars are protruding above, or recessed below, the surface of the resist layer 516) is planarized to completely level the tops of the nanopillars 536c with the top surface of the resist layer 516. This planarization step may also be used to adjust the height of the overall structure. As mentioned in the context of FIG. 3A, planarization has certain advantages such as providing a clean, even top to each nanopillar for binding of the biomolecular bridge, and for providing a blockade to sagging of the biomolecular bridge molecule.

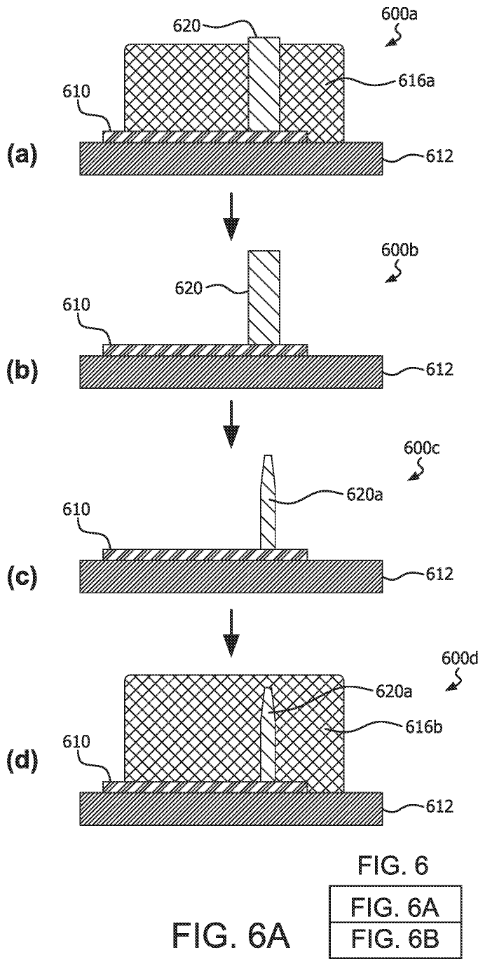

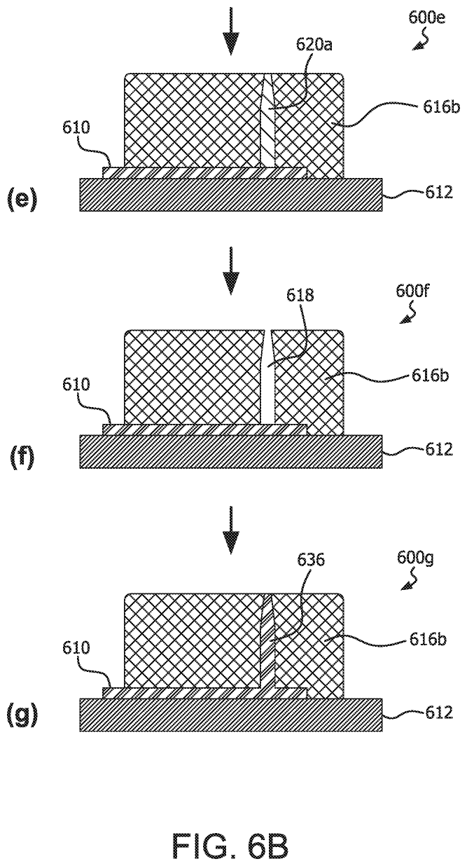

FIG. 6 illustrates additional embodiments of a lithographic method designed to provide nanopillars, comprising use of a sacrificial metal nanopillar for tapering of vertical holes. The method illustrated in FIGS. 6A and 6B proceeds through structures 600a-600g, beginning with the wider cylindrical nanopillars of structure 1d in FIG. 1, and ending in substantially narrower nanopillars of structure 600g in FIG. 6B, with the structures illustrated in cross section for clarity.

With reference to FIG. 6A, beginning structure 600a is essentially the equivalent of structure 1d in FIG. 1, wherein a substantially cylindrical nanopillar 620 was grown by deposits into nanopatterned holes formed in a resist layer 616a covering both the underlying electrodes 610 previously deposited on substrate 612. The cylindrical nanopillar 620 provided in this example may have a diameter of about 20 nm to about 50 nm, recognizing that the initial metal nanopillar is sacrificial. The height of the nanopillars 620 may be from about 3 nm to about 100 nm.

With reference now to structure 600a, a PMMA layer 616a covering electrode 610, the electrode previously deposited onto substrate 612, was patterned with a vertical hole through the PMMA layer 616a and down to the electrode using e-beam or nanoimprint lithography, and the resulting hole was filled with copper (Cu) to obtain the sacrificial Cu nanopillar 620. For clarity, just one nanopillar 620 is illustrated in this method, recognizing that in practice, an array of electrode pairs is preferred, wherein the nanoimprinting would result in pairs of holes aligned with the pairs of electrodes. In various embodiments, other metals may be used as the sacrificial nanopillar 620, such as nickel (Ni). In structure 600a, and as discussed above in the context of other methods, the nanopillar 620 comprises a vertical post extending from the surface of the horizontally disposed electrode 610 up to about level with the top surface of the PMMA layer 616a. However, in this case it is preferable that the electrode 610 and the sacrificial nanopillar 620 not comprise the same material, since the nanopillar 620 will be dissolved away without damage to the underlying electrode 610. For example, the underlying electrode 610 may comprise Al, Ru, Pt, Pd, or Au, whereas the sacrificial nanopillar 620 may comprise Cu or Ni.

With continued reference to FIG. 6A, the PMMA layer 616a in structure 600a is dissolved away to expose the bare Cu sacrificial nanopillar 620 attached to the underlying electrode 610. The sacrificial nanopillar 620 may have a diameter of from about 20 nm to about 50 nm. The next step in the method is to reduce the diameter of the Cu nanopillar 620 by chemical or RIE etching to produce a narrower Cu nanopillar 620a having an average diameter of about 5 nm, as shown in structure 600c. The etching is conducted so as to provide a tapered shape to the narrowed nanopillar 620a as shown. The step to convert structure 600c to 600d comprises casting a new PMMA layer 616b to cover the diameter-reduced Cu nanopillar 620a.

With reference to FIGS. 6A and 6B, the step to convert structure 600d to 600e comprises planarization horizontally across the PMMA layer 616b, such as by RIE, to ensure the entire top of the narrowed Cu nanopillar 620a is exposed. As shown by structures 600e and 600f, the Cu nanopillar 620a is then dissolved away to produce a new hole 618 in the PMMA layer 616b. This new hole 618 is then filled in with Ru, Pt, Pd, or Au (or other metal or semiconducting material) to provide new nanopillar 636, optionally matching the material of the underlying electrode 610. The resulting structure 600g can be optionally planarized again, and/or subjected to any of the manipulations exemplified in FIG. 3, prior to exposure to a solution of bridge molecules. In various embodiments, the exposed top of the nanopillar 636 in structure 600g has a diameter of about 5 nm, promoting attachment of only a single, or at most just a few, biomolecules. Structure 600g (recognizing that the structure illustrated is one half of a pair of such structures, and there is typically an array of pairs of such structures on a chip) is usable in a solid state molecular sensor further comprising a molecular bridge molecule (oligonucleotide, diazonium-enhanced, polypeptide, α-helix GBP, etc.) bridging across each pair of narrowed nanopillars 636, as discussed above in the context of FIG. 4.

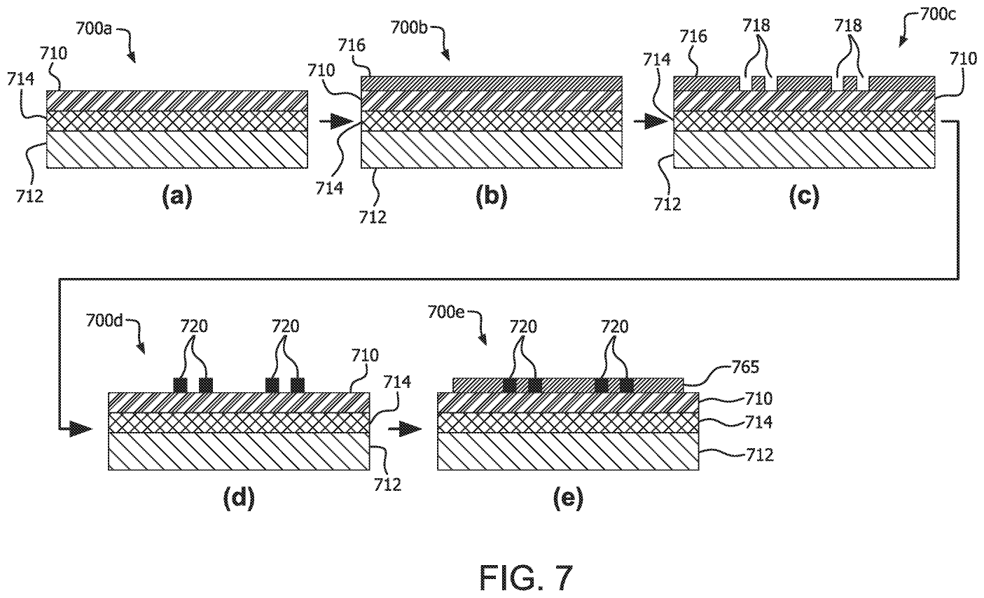

The method depicted in FIG. 7 is a variation of the method in FIG. 1, using different materials. FIG. 7 illustrates embodiments of a nanopillar array fabrication process on CMOS-compatible Cu films. For electroless deposition of metal nanopillars into vertical holes, a Cu or Ni base layer is desirable. Cu is more CMOS compatible that a Ni base layer.

As shown in FIG. 7, a chip 700a comprises a Cu/Ti film layer 710 on a Si substrate 712 that further comprises a Sift layer 714. The first step of the method comprises spin-coating a PMMA layer 716 overtop the metal film layer 710, to arrive at structure 700b. Then, the PMMA layer is nanopatterned using e-beam lithography or nanoimprint lithograph, giving rise to the array of vertical holes 718 in the PMMA layer 716, as shown in structure 700c. Metal is then deposited in the holes by e-beam evaporation deposition, and subsequent lift-off of the resist layer, producing structure 700d comprising nanopillars 720 in electrical contact with the metal film layer 710. Lastly, a silica layer 765 is then deposited and the final construct planarized to provide structure 700e having metal nanopillars 720 with only the top circular surface exposed.

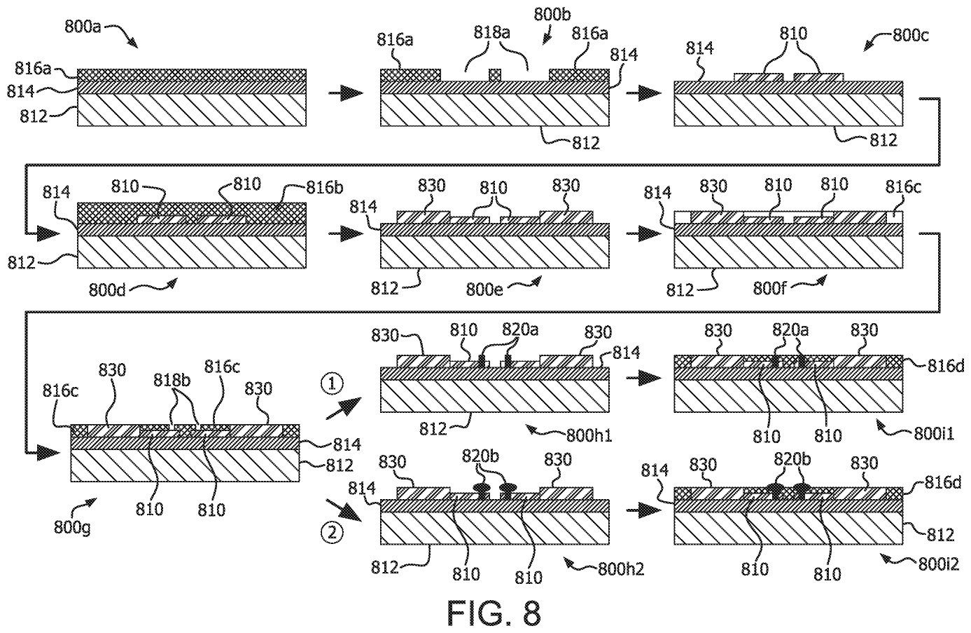

FIG. 8 sets forth various embodiments of nanofabrication steps for providing nanopillar arrays on circuit chip devices, comprising either physical vapor deposition (sputtering or evaporation), electrodeposition, or electroless deposition of metal. The method illustrated in FIG. 8 splits into optional pathways “1” and “2,” as explained further below.

The method of FIG. 8 begins with structure 800a comprising a three layer device wherein a PMMA layer 816a is spin-coated onto a substrate comprising a Si layer 812 and a SiO₂ layer 814. The next step in the method comprises a first e-beam lithography process to introduce openings 818a into the PMMA resist layer, as shown in structure 800b. The openings 818a are rectangular, measuring about 200 nm long and 30-50 nm wide, and spaced apart by a nanogap of about 10-30 nm. To convert structure 800b into 800c, a Cu metal deposition and lift-off process is used to leave behind a pair of ˜200 nm spaced apart Cu electrodes 810 on the substrate. In the next step, a photoresist layer 816b is spin-coated on top of the pair of Cu electrodes 810, as shown in structure 800d. Next, a second round of lithographic patterning, metal deposition, and lift-off provides structure 800e, wherein microelectrodes 830 are disposed in physical and electrical contact with the Cu nanoelectrodes 810. In the next step of the method, another PMMA layer 816c is spin-coated over the sets of microelectrodes 830 and nanoelectrodes 810, as shown in structure 800f. To arrive at structure 800g, nanopatterning is used to produce holes 818b in the PMMA layer.

At this point in the method illustrated in FIG. 8, one of two routes may be taken, beginning with structure 800g. In the route marked “1,” e-beam evaporation deposition and lift-off provides nanopillars 820a in the nanopatterned holes. To arrive at a structure usable for a sensor, structure 800h1 is then coated with an insulation layer 816d and planarized to arrive at structure 800i1, wherein the heights of the microelectrodes 830 and the nanopillars 820a are even. Of note is that the nanoelectrodes 810 can remain buried underneath the insulation layer 816d because only the exposed nanopillars 820a are needed for bridging a biomolecule. In the route marked “2,” methods discussed in the context of FIG. 3 may be employed to ultimately arrive at structure 800i2 comprising extended nanopillar deposits 820b shown protruding above and horizontally across the planarized PMMA layer 816d. The structure 800i2 is then usable in a molecular electronics sensor by bridging a single biomolecule or at most just a few biomolecules across the spaced apart nanopillars 820b.

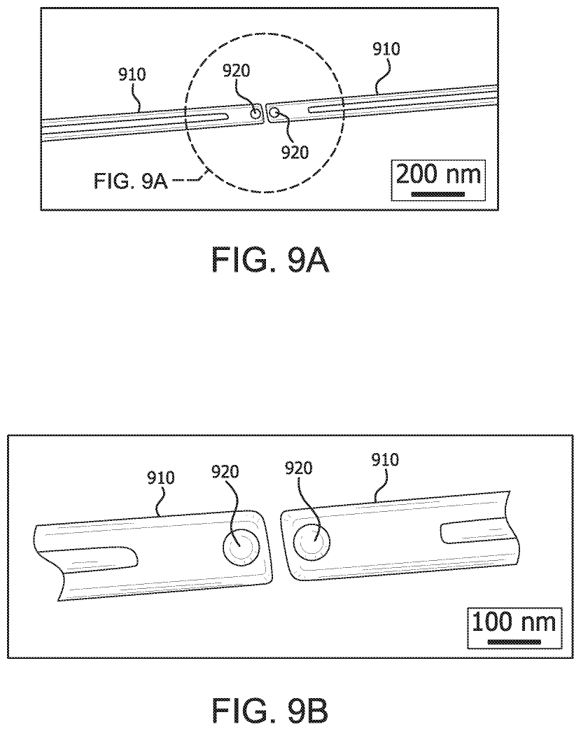

FIGS. 9A and 9B are drawings of actual SEM micrographs taken of nanopillars 920 produced on a pair of spaced-apart electrodes 910 by sputter-depositing and lift-off processes. FIG. 9A is a drawing of a lower magnification SEM taken in a tilted view and FIG. 9B is a drawing of an SEM at higher magnification, as shown by the nm scales in each drawing. The resulting nanopillars 920 were produced on CMOS compatible Cu metal films, wherein each nanopillar 920 comprises an exposed top surface measuring about 30 nm in diameter. The structure was prepared on a sequencing electrode chip, wherein both the Cu and Au metals can be deposited by sputter deposition, electroless deposition or electrochemical deposition. The pair of exposed 30 nm diameter circular tops of the nanopillars 920 allow attachment of one or a limited number of bridge molecules, such as DNA oligonucleotide, for sensor bridge formation. with the, and disclosed herein. FIG. 9A shows a tilted view of a pair of electrodes 910 comprising nanopillars 920.

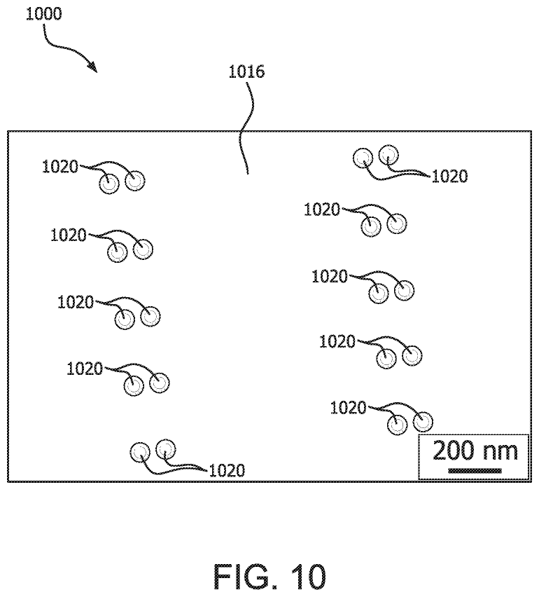

FIG. 10 is also a drawing of an actual SEM micrograph of an array 1000 of nanopillar top surfaces 1020. In essence, FIG. 10 is the reduction-to-practice of the array 200 depicted in FIG. 2, with some nuances to the insulative/resist layer. Herein, the nanopillar top surfaces 1020 are seen exposed flush with a 50 nm tall Sift dielectric layer 1016. The Au nanopillar array 1000 was formed by resist nanopatterning (e-beam lithograph or nanoimprinting) to form vertical nanosized holes into which Au electrode material was sputter-deposited and lift-off processed. In other embodiments, the Au nanopillars may be created by electroless or electrochemical deposition. The Sift planarized top layer 1016 was prepared by HSQ resist spin-coating and annealing at 150° C. to convert the layer to Sift, followed by planarization using Sift etch RIE processing in a CHF₃ and Argon mixed gas at 100 Watts power for 85 seconds.

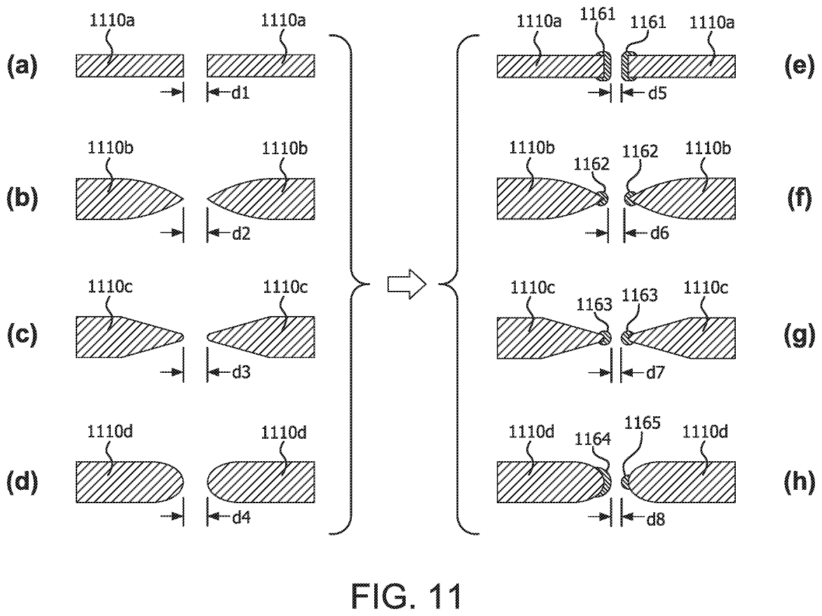

FIG. 11 illustrates top views of various nanoelectrode geometries, having different shapes and features, and how starting electrode geometries may be modified by depositing additional metal on the electrodes at the portions of the electrodes facing one another. Substrate materials, such as Si with a SiO₂ surface insulator layer, are not shown in these drawings of electrode pairs for clarity.

On the left side of FIG. 11, beginning electrode pairs (a), (b), (c) and (d) are shown, each with starting nanogaps having distance d1, d2, d3 and d4, respectively. These types of electrode pairs may be made by nanoimprint lithography for scaled-up manufacturing, or e-beam lithography, and/or by other means. The pair of electrodes 1110a comprise a pair of rectangular electrodes, made of Al, Cu, Ru, Pt, Pd, or Au or other metals. The pair of electrodes 1110b have a spearhead shape, so as to remove local electrical current concentrations that can occur during electrodeposition of Au. The pair of electrodes 1110c comprise tapered electrodes further comprising rounded tips, so as to provide slightly less electric current concentration for slightly larger diameter Au deposits having better adhesion to the electrodes. The pair of electrodes 1110d comprise fully rounded electrodes. As may be the case, any of these pairs of electrodes (a), (b), (c), and (d) might have shapes prone to binding to multiple biomolecules, and/or inappropriate gap distances, d1, d2, d3, d4 to promote binding of a biomolecule of particular length across the gap. In this regard, FIG. 11 illustrates various embodiments of electrode modification that succeed to reduce the surface area for biomolecular bridge binding and to adjust the gap distance between spaced-apart electrodes.

With continued reference to FIG. 11, in the conversion of electrode pair (a) to corresponding pair of electrodes (g), the gap d5 is adjusted by the degree and time of Au electroplating that produces the Au deposits 1161 on adjacent edges of the electrodes 1110a. In this way, the nanogap distance d5 is less than starting gap distance d1, and the gap distance d5 may be closed by extended depositing of Au, such as down to a gap of from about 3 nm to about 10 nm. Similarly, for the spearhead shaped electrodes 1110b, Au depositing provides Au plated tips 1162 at the points of the electrodes 1110b, wherein the gap distance d6 is less than the beginning gap distance d2, and is further shortened by extended Au depositing. Likewise, for the rounded tip electrodes 1110c, Au depositing provides Au plated tips 1163 at the rounded tips of the electrodes 1110c, wherein the gap distance d7 is less than the beginning gap distance d3, and is further shortened by extended Au depositing.

With further reference to FIG. 11, the modification of electrode pair (d) into electrode pair (h) illustrates that the Au depositing on each electrode 1110d in a pair of electrodes need not be symmetrical. As discussed above, electrodepositing can comprise direction-guided electrodeposition, such that one electrode receives the Au depositing. The configuration in (h) can be obtained by direction-guided electrodeposition on the right electrode 1110d followed by an annealing process to provide the balled-up Au deposit 1165. Afterwards, direction-guided electrodeposition can be used with the reverse polarity to preferentially deposit Au on the left electrode 1110d, which can be left as a broader deposit 1164. In each of these methods in FIG. 11, extended deposition of Au can be used to close down the gap distance d5, d6, d7, and d8 as needed.

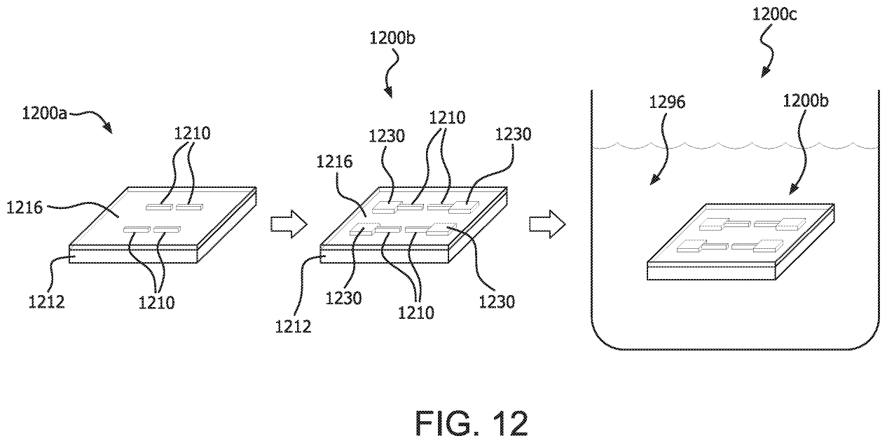

FIG. 12 illustrates embodiments of a nanoelectrode fabrication process, related to the steps in FIG. 8, and in particular, electrodeposition steps in routes 1 and 2 in FIG. 8. FIG. 12 demonstrates the concept of nanoelectrode gap control by electrochemical deposition (e.g., electroless deposition or electrodeposition), for the purpose of “length matching” electrode gap distances with the length of a particular bridge molecule desired for use, or for more controlled and precise sensor bridge formation. In the method of FIG. 8, use of a Cu or Ni base layer has been demonstrated in nanogap control by electrodeposition, Cu being used as the base layer for CMOS compatibility rather than Ni. This innovative process enables predictable setting of the electrode nanogap, such that the desired bridge molecule, having particular chemistry and known length, can be optimally used in reproducible bridge formation across tuned nanogap distances.

FIG. 12 shows that a chip 1200a can first be configured with Cu nanoelectrodes 1210 as per steps (a), (b), (c) and (d) in FIG. 8. The chip 1200a comprises Cu nanoelectrodes on a Si substrate 1212, and further comprising a Sift dielectric layer 1216 covering the nanoelectrodes 1210. In other words, in various embodiments, chip 1200a may be substantially similar to structure 800d in FIG. 8. Chip 1200a can then be modified into chip 1200b by metal deposition and lift-off to provide microelectrodes 1230, and spin-coting of another PMMA layer 1216 overtop. After the second e-beam lithography (e.g., what was described to produce the holes 818b in structure 800g in FIG. 8), the chip 1200b can then be subjected to electrodeposition as illustrated schematically by the arrangement 1200c, wherein the electrode array chip 1200b is exposed to an electrodeposition electrolyte 1296.

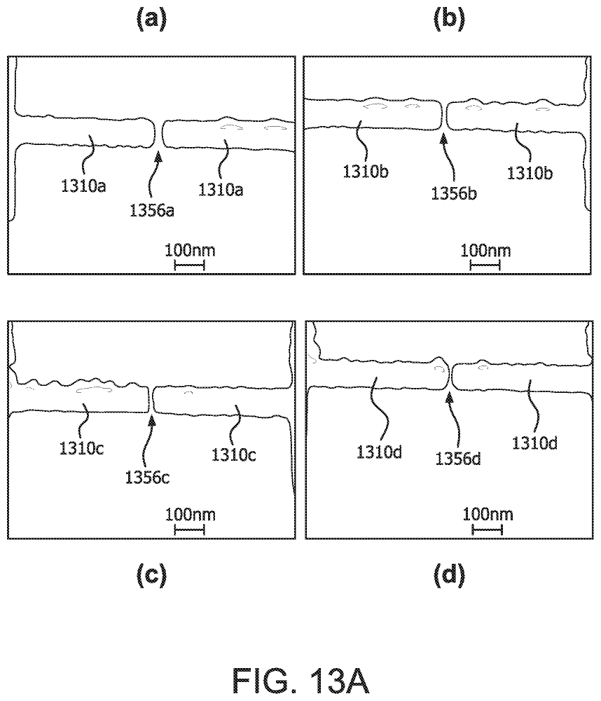

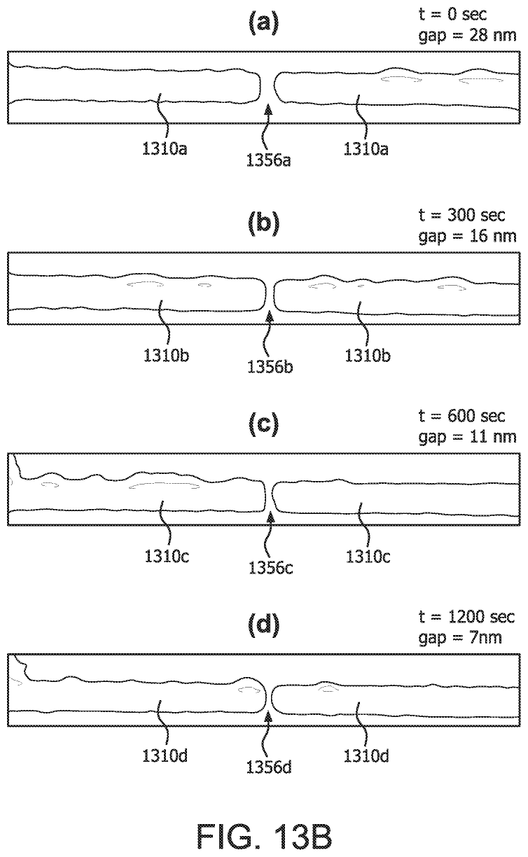

FIG. 13 illustrates various embodiments of a method of controlling nanogap distances, the method reduced-to-practice using Au depositing over time on a rectangular Au electrode pair at 90° C., pH=8, reducing the spacing of the electrode gap as a function of deposition time. The sketches in FIGS. 13A and 13B are line drawings of actual SEM micrographs. The drawings of SEM micrographs in (a)-(d) in FIG. 13A show the 100 nm scale on the original micrographs. FIG. 13B are drawings of the same SEM micrographs drawn in FIG. 13A, but with a focus on just the electrode gap region, without change in magnification. Beginning with (a) in FIG. 13A, at time t=0, the electrode gap 1356a between electrodes 1310a was about 28 nm. This electrode gap was the result of the lithographic method used to deposit the rectangular Au electrode pair. As shown in (b), Au electroless deposition of Au for 300 seconds resulted in a closing of the nanogap 1356b between electrodes 1310b to about 16 nm. As shown in (c), Au electroless deposition of Au for 600 seconds resulted in a closing of the nanogap 1356c between electrodes 1310c to about 11 nm. Lastly, and as shown in (d), Au electroless deposition of Au for 1200 seconds resulted in a closing of the nanogap 1356d between electrodes 1310d to about 7 nm. FIG. 13B reiterates the results of the experiment in a more organized way, with the time duration and nanogap measurement directly next to the corresponding SEM micrograph representation. It is evident from the data that a selected Au electroless deposit time dictates the nanogap distance.

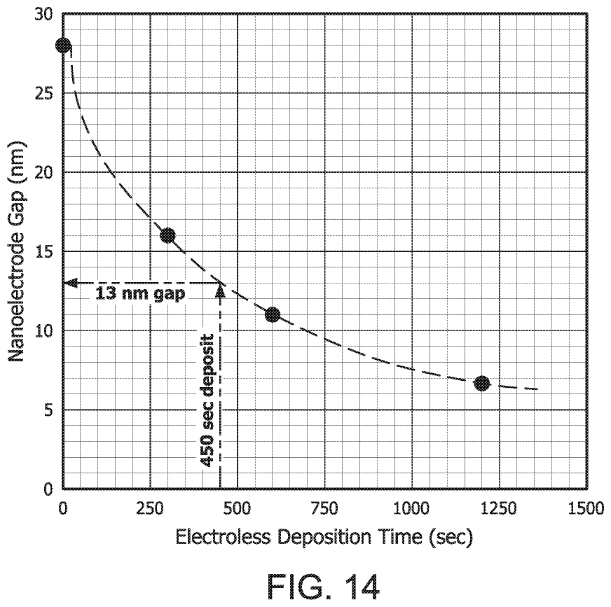

The data from the experiment summarized in FIGS. 13A-13B can be plotted in an x/y plot. FIG. 14 is a plot of the nanoelectrode gap (in nm) versus the electroless deposition time (in seconds). The four (4) data points are the data from FIG. 13. These data points can be fit to a curve as shown in FIG. 14, giving rise to a calibration curve wherein a desired nanoelectrode gap can be interpolated to an approximate time one would use in electroless Au deposition to obtain that desired nanogap. So, for example, using the x/y plot in FIG. 14 for such an interpolation as shown by the dashed arrows in the plot, if one desired a 13 nm gap, such as to accommodate bridging of a biomolecule having a functional length of about 13 nm, one would conduct the electroless Au depositing for about 450 seconds. In this way, one can “dial-in” the desired nanogap dimension by setting the deposition time. A similar x/y plot can be constructed by gathering deposition data for other metal depositing, such as Ru, Pt, or Pd.

Methods, apparatus and system for preparing nanopillar structures for electrodes in molecular sensors are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for a composition or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A structure for use in a molecular electronics sensor, the structure comprising:
a pair of nanoelectrodes disposed on a substrate and comprising a first metal, each pair of nanoelectrodes comprising a first nanoelectrode and a second nanoelectrode spaced 5 apart from the first nanoelectrode by a nanogap,
a resist or dielectric layer covering the pair of nanoelectrodes and the nanogap; and
a pair of nanopillars comprising a second metal, each pair of nanopillars comprising a first nanopillar and a second nanopillar spaced-apart from the first nanopillar by a nanopillar gap,
wherein a bottom surface of the first nanopillar is physically and electrically connected to the first nanoelectrode, and a bottom surface of the second nanopillar is physically and electrically connected to the second nanoelectrode, and
wherein the first and second nanopillars each comprise posts projecting substantially vertically through the resist or dielectric layer such that only a top surface of each nanopillar is uncovered by the resist or dielectric layer.

2. The structure of claim 1, wherein the top surface of each nanopillar is: (a) protruding beyond a top surface of the resist or dielectric layer; (b) flush with the top surface of the resist or dielectric layer; or (c) recessed below the top surface of the resist or dielectric layer.

3. The structure of claim 1, further comprising a bridge molecule having a first end and a second end, the first end of the bridge molecule bonded to the first nanopillar and the second end of the bridge molecule bonded to the second nanopillar, bridging the nanopillar gap.

4. The structure of claim 1, wherein the first metal comprises Al, Cu, Ru, Pt, Pd, or Au, and the second metal comprises Ru, Pt, Pd, or Au.

5. The structure of claim 1, wherein the first metal comprises Al and the second metal comprises Ru.

6. The structure of claim 1, wherein the top surface of at least one nanopillar in the pair of nanopillars comprises a mushroom protrusion extending the nanopillar horizontally over a portion of a top surface of the resist or dielectric layer.

7. The structure of claim 1, wherein only one nanopillar in the pair of nanopillars further comprises a horizontal portion extending across a portion of a top surface of the resist or dielectric layer and toward the other nanopillar in the pair of nanopillars.

8. The structure of claim 1, wherein at least one nanopillar in the pair of nanopillars comprises a vertically tapered nanopillar, and wherein a bottom portion of the vertically tapered nanopillar is larger in diameter than a top portion of the vertically tapered nanopillar.

9. The structure of claim 8, wherein both nanopillars in the pair of nanopillars comprise vertically tapered nanopillars.

10. A method comprising:
depositing a pair of nanoelectrodes on a substrate, the pair of nanoelectrodes comprising a first metal and including a first nanoelectrode and a second nanoelectrode spaced-apart from the first electrode by a nanogap;
applying a resist coating to form a resist layer over the pair of nanoelectrodes and the nanogap, the resist layer having a horizontal exposed top surface;
patterning a pair of open holes vertically through the resist layer, the patterning comprising one hole per nanoelectrode, each hole beginning with an exposed portion of the nanoelectrode and extending vertically from the nanoelectrode through the resist layer, ending in an opening at the horizontal exposed top surface of the resist layer; and
depositing a second metal into each hole to form a pair of nanopillars, each nanopillar formed in the shape of the hole, the nanopillar having a bottom portion in physical and electrical contact with the nanoelectrode and an exposed top surface near, at, or protruding above the horizontal exposed top surface of the resist layer.

11. The method of claim 10, wherein the substrate comprises a Si layer and a SiO₂ insulative layer onto which the nanoelectrodes are deposited.

12. The method of claim 10, further comprising the step of planarizing the horizontal exposed top surface of the resist layer after the step of depositing the second metal such that the exposed top surface of each nanopillar is flush with the horizontal exposed top surface of the resist layer.

13. The method of claim 12, wherein the exposed top surface of each nanopillar comprises a circular shape.

14. The method of claim 12, further comprising the step of bonding a bridge molecule between the pair of nanopillars, such that a first end of the bridge molecule is bonded to one nanopillar and a second end of the bridge molecule is bonded to the other nanopillar in the pair of nanopillars.

15. The method of claim 12, wherein the depositing of second metal is continued for a time sufficient to produce a mushroom protrusion on the top surface of each nanopillar extending vertically above and horizontally across a portion of the horizontal exposed top surface of the resist layer.

16. The method of claim 12, further comprising, after the step of depositing the second metal, the step of direction-guided electrodeposition of additional second metal on one nanopillar creating a horizontally disposed portion on the one nanopillar extending across the horizontal exposed top surface of the resist layer in a direction toward the other nanopillar in the pair of nanopillars.

17. The method of claim 10, further comprising, after the step of patterning the pair of open holes, the step of adding resist coating into a top portion of each of the patterned open holes to reduce the size of each opening of each hole.

18. The method of claim 10, further comprising, after the step of depositing the second metal, the additional steps of:
dissolving away the resist layer to leave exposed nanopillars;
reducing the diameter of and optionally vertically tapering each nanopillar by an etching process;
casting a new resist layer to entirely cover the nanopillars;
planarizing the resist layer such that a top surface of each nanopillar is flush with a top surface of the resist layer;
dissolving away each nanopillar to leave behind a hole;
depositing a material into each hole to create nanopillars physically and electrically attached to the nanoelectrodes.

19. The method of claim 18, wherein the first metal comprises Al, Cu, Ru, Pt, Pd or Au, the second metal comprises Cu or Ni, and the material comprises Ru, Pt, Pd or Au.

20. A method of nanofabrication comprising:
depositing a pair of nanoelectrodes on a substrate, the pair of nanoelectrodes comprising a metal or semiconducting material and including a first nanoelectrode and a second nanoelectrode spaced-apart from the first nanoelectrode by a first nanogap;
choosing a second nanogap having distance less than the first nanogap,
determining an electroless deposition duration time required to narrow the first nanogap down to the second nanogap by interpolating the second nanogap on an x/y plot of nanogap distance versus electroless deposition duration time; and
preforming electroless deposition of a metal or noble metal on the nanoelectrodes for the electroless deposition duration time thus determined, producing the second nanogap between the nanoelectrodes.
